(19) United States
(12) Patent Application Publication
(10) Pub. No.: US 2025/0375149 A1
(43) Pub. Date: Dec. 11, 2025

(54) METHOD FOR IMPROVING THE REAL-WORLD SKIN SENSITIZATION IMPACT OF FORMULATED PRODUCTS

(71) Applicant: Kenvue Brands LLC, Summit, NJ (US)

(72) Inventors: Ladan Fakhrzadeh, Skillman, NJ (US); Evren Atillasoy, Fort Washington, PA (US); James P. Bowman, Loveland, OH (US); Michael Cork, Sheffield (GB); Alain Khaiat, Heritage View (SG); Tony J. McGuire, Skillman, NJ (US); Otto Mills, Doylestown, PA (US); Teginder Singh, Skillman, NJ (US)

(21) Appl. No.: 18/876,799

(22) PCT Filed: Jul. 7, 2023
(86) PCT No.: PCT/IB2023/057039

§ 371 Date: Dec. 19, 2024

Related U.S. Application Data
(60) Provisional application No. 63/359,077, filed on Jul. 7, 2022.

(51) Int. Cl. A61B 5/00; G16H 10/40

(52) U.S. Cl. CPC A61B 5/411; A61B 5/7275; G16H 10/40

(57) ABSTRACT

A method of predictively assessing skin sensitization of a personal care product formulation includes: calculating an upper confidence limit at a desired confidence level from multiple physical tests for skin sensitization of each of the ingredients in the personal care product formulation to define calculated skin sensitization values for the respective ingredients; applying at least one safety factor to at least one of the calculated skin sensitization values for the ingredients to define adjusted skin sensitization values for the respective ingredients; summing the adjusted skin sensitization values of the ingredients to define a total sensitization of the personal care product formulation; and comparing the total sensitization to a target product sensitization for the personal care product formulation. Such personal care product formulations do not require further testing and are capable of application to skin by users. Predictive systems and methods of preparing a personal care products are also disclosed.

### FIELD OF THE INVENTION

The present invention is related to personal care products and methods for formulating the same. In particular, the present invention is related to methods of predictively assessing skin sensitization of personal care product formulations, and systems for the same.

### BACKGROUND OF THE INVENTION

Skin sensitization phenomenon consists of induction of sensitization and subsequent elicitation of an allergic response on the skin of a human patient or user of a personal care product. Sensitization potential is dependent on bioavailability or absorption of a chemical as well as the initial and rate limiting ability of the chemical to bind epidermal proteins to elicit the response. Human repeat insult patch testing (HRIPT) on cosmetic finished product formulations has been used as one part in skin sensitization assessment potential. Such testing, by itself, however, is time consuming as the time for product testing results may be in the order at least eight weeks or more. Further, if there is a reaction on this product testing, then ingredient-by-ingredient testing may be required.

Given that a finished product formulation is often a complex blend of varying chemistries, the risk assessment process for skin sensitization may involve consideration of the influence of formulation components (matrix effects) on the bioavailability and sensitizing potential of the ingredient (ingredient sensitization effect), as well as the finished product delivery system.

Thus, there is a need in the art for improved tools for evaluating sensitization potential due to the complex nature of personal care products.

### SUMMARY OF THE INVENTION

The present invention is directed to a method for developing a product having an improved skin sensitization risk assessment impact, and the resulting product developed as a result of the method. The method may evaluate skin sensitization hazard for ingredient components in formulated products and may identify improvements in skin sensitization risk assessment safety process based on those evaluations. The method of the present invention includes HRIPT test results, but the methodology of the present invention is further based on a binomial distribution of sample size and percent of subjects in the population who would develop sensitization under identical or suitable test conditions. The statistical probability of detecting population sensitivity and reliability for a finished cosmetic formulation under identical parameters improves with a larger HRIPT sample or population size.

Cosmetic finished product formulations commonly include of a desired set of ingredients with a defined dose intended to deliver a pertinent function such as stability, micro, aesthetics, and/or to deliver a certain attribute. Given that a finished product formulation is a complex blend of varying chemistries, the risk assessment process for skin sensitization demands the consideration of the influence of formulation components (matrix effects) on the bioavailability and sensitizing potential of the ingredient (ingredient sensitization effect), as well as the finished product delivery system.

The method of the present invention provides a real-world consumer sensitization threshold with a 95% upper limit confidence score application for ingredient chemistries in a finished product formulation and encourages formulators to move toward designing finished formulations with improved safety confidence. This method may be utilized to track and improve skin sensitization risk assessment performance of a product line, brand, product function (e.g., shampoo, lotion, serum, wash, soup, mask) or product type (e.g., rinse off, leave on) through use of ingredient's sensitization thresholds and upper limit 95% confidence scores for a real-world consumer use of a finished product formulation. Metrics derived from ingredient score tests of common set of ingredients across various formulation types over time may be used to predict upper limit confidence formulation sensitization threshold for real-world consumer use of a finished product and can identify organization-wide formulation design changes needed to strengthen and improve future skin sensitization threshold risk assessment predictions.

In one aspect of the present invention, a method of predictively assessing skin sensitization of a personal care product formulation including a plurality of ingredients, includes:
calculating an upper confidence limit at a desired confidence level from multiple physical tests for skin sensitization of each of the ingredients in the personal care product formulation in order to define calculated skin sensitization values for the respective ingredients;
applying at least one safety factor to at least one of the calculated skin sensitization values for the ingredients to define adjusted skin sensitization values for the respective ingredients;
summing the adjusted skin sensitization values of the ingredients to define a total sensitization of the personal care product formulation; and
comparing the total sensitization to a target product sensitization for the personal care product formulation, wherein the product formulation for the personal care product formulation does not require further testing and is capable of application to skin by users when the total sensitization is less than the target product sensitization.

The skin sensitization of each of the ingredients may be defined as the percentage of subjects having an allergic response to physical allergic tests, such as human repeat insult tests. Thus, low values of skin sensitization are desired.

The method may further include: setting an initial sensitization target for the personal care product formulation; and obtaining the skin sensitization of each of the ingredients from a computer database having multiple test results for each ingredient. The initial sensitization target may be about 0.2 percent or less than 0.2 percent, and the target product sensitization may be about 0.1 percent or less than 0.1 percent.

The method further includes selecting ingredients that have been used in multiple personal care products, for example, about ten or more than ten different personal care products. Further, it is desirable ingredients being selected have been tested on a plurality of subjects, such as about 2000 subjects or greater than 2000 subjects.

With such a large pool of testing results, sensitization values are determined at confidence level of at least 95%. Instead of using the mean value at this confidence level, the upper confidence limit is used to provide more conservative predictive methodology. The ingredient sensitization values and/or the ingredient upper confidence limit may be adjusted for the weight composition of the ingredients in the proposed formulations. The methods set forth herein, however, do not require such weight adjustments as typical formulations often contain a large plurality of proposed ingredients.

The methods described herein may further include applying a product usage factor to the calculated skin sensitization values for the ingredients; where the product usage factor may be based on an estimated amount of product to be used and an estimated area of product use. For example, the product usage factor may be a ratio of an estimated daily use (which may be in grams or milligrams) over an estimated application surface area (which may be in square centimeters or square millimeters).

The step of applying at least one safety factor may include multiplying the at least one calculated skin sensitization value for at least one ingredient by the at least safety factor. The safety factor may be applied or multiplied to multiple ingredients, include all or substantially all of the ingredients. Safety factors may include safety factors based on (a) impairment to skin barrier function, (b) based on product delivery effecting skin occlusion and/or based on occlusive nature application site, (c) based potential for pre-conditioned skin having potential for activated inflammatory response, and combinations thereof. The order of the applied safety factors is not critical, and any order of applied safety factors may suitably be used. If applied, the safety factor based on impairment to skin barrier function is about 5 or greater than 5; if applied, the safety factor based on product delivery effecting skin occlusion and/or based on occlusive nature application site is about 5 or greater than 5; and, if applied, the safety factor based potential for pre-conditioned skin having potential for activated inflammatory response is about 2 or greater than 2.

In another aspect of the present invention, a system to predictively assess skin sensitization of a personal care product formulation including a plurality of ingredients is provided. The system may include:
a computing device;
a memory storing instructions that, when executed by the computing device, cause the computing device to perform operations comprising:
calculating an upper confidence limit at a desired confidence level from multiple tests for skin sensitization of each of the ingredients in the personal care product formulation in order to define calculated skin sensitization values for respective ingredients;
applying at least one safety factor to the calculated skin sensitization values for the ingredients to define adjusted skin sensitization values for the respective ingredients;
summing the adjusted skin sensitization values of the ingredients to define a total sensitization of the personal care product formulation; and
comparing the total sensitization to a target product sensitization for the personal care product formulation, wherein the product formulation does not require further testing and is capable of application to skin by users when the total sensitization is less than the target product sensitization.

In yet another aspect of the present invention, a method of preparing a personal care product may include:
selecting a plurality ingredients for use in a personal care product formulation;
calculating an upper confidence limit at a desired confidence level from multiple physical tests for skin sensitization of each of the ingredients in the personal care product formulation in order to define calculated skin sensitization values for the respective ingredients;
applying at least one safety factor to at least one of the calculated skin sensitization values for the ingredients to define adjusted skin sensitization values for the respective ingredients;
summing the adjusted skin sensitization values of the ingredients to define a total sensitization of the personal care product formulation;
comparing the total sensitization to a target product sensitization for the personal care product formulation; and
if the total sensitization is less that the target product sensitization, combining the ingredients to form the personal care product, wherein the product formulation for the personal care product formulation does not require further testing and is capable of application to skin by users.

These and other aspects, features and advantages of the present invention will become apparent from the following detailed description of illustrative embodiments thereof, which is to be read in connection with the accompanying drawings. Corresponding reference element numbers or characters indicate corresponding parts throughout the several views of the drawings.

### BRIEF DESCRIPTION OF THE DRAWINGS

FIG. 1 is a flowchart for predictively assessing skin sensitization of a personal care product formulation accord to the present invention.

FIG. 2 is a flowchart presenting additional details of portion of the flowchart of FIG. 1.

FIG. 3 is a flowchart presenting additional details of another portion of the flowchart of FIG. 1.

FIG. 4 is a schematic illustration system for predictively assessing skin sensitization of a personal care product formulation accord to the present invention.

FIG. 5 is a block diagram of a general computer system.

### DETAILED DESCRIPTION OF THE INVENTION

The present invention is a method for predictively assessing skin sensitization of a personal care product formulation. The method is particularly useful in that it allows for personal care product formation without requiring additional physical, such as HRIPT, testing for skin sensitization.

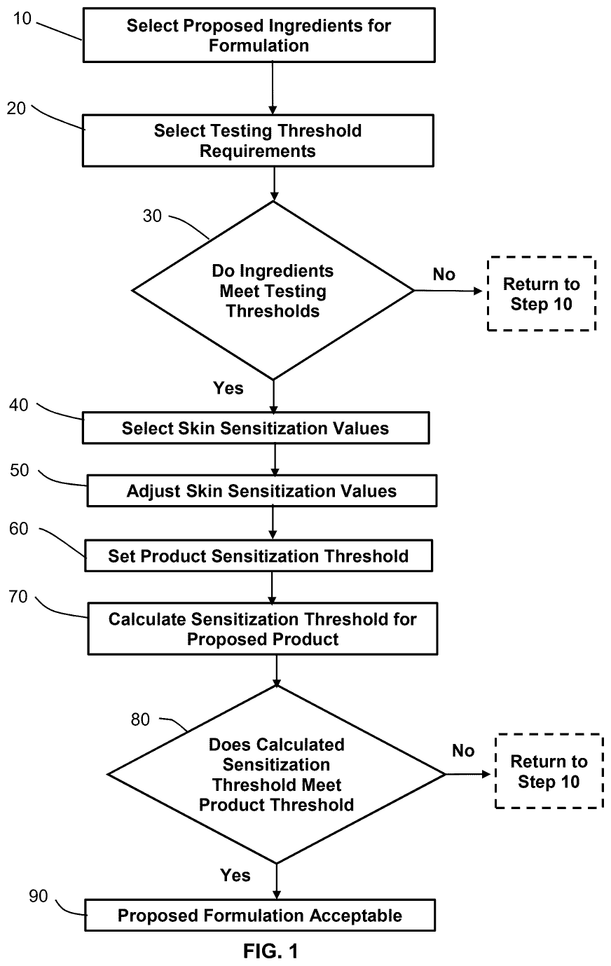

FIG. 1 is a flowchart for predictively assessing skin sensitization of a personal care product formulation accord to the present invention. As illustrated in FIG. 1, proposed ingredients are first proposed at step 10. The ingredients, types and amounts thereof, may be based on the end use demands of the final product, such as but not limited to product function (e.g., shampoo, lotion, serum, wash, soup, mask), product type (e.g., rinse off, leave on), and the like.

At step 20 only ingredients meeting certain threshold conditions are selected. It is desirable to set the threshold conditions such that the predictive assessment is representative of real-world experience. For example, it may be desirable to select ingredients having a physical test history (e.g., HRIPT) in a plurality of finished personal care product formulations and across a large plurality of subjects tested. Further, it may be desirable to only select those ingredients meeting a sensitization value or criteria.

The plurality of finished personal care product formulations may be set at any reasonable value, such as ten or more formulations. One reason for selecting a plurality of finished products is to guard against unexpected adverse effects as different ingredients are being formulated in different products. The plurality of ten or more formulations is non-limiting, and other plurality values may be used. Such other values may include five or more formulations, fifteen or more formulations, twenty or more formulations, fifty or more formulations, and the like.

The number of subjects tested may be set at about 2,000 or more subjects. The present invention, however, is not so limited. The number of subjects tested may be set at 500 or more subjects; 1,000 or more subjects; 3,000 or more subjects; 5,000 or more subjects; and the like. While there is no set upper limit for the number of subjects to be tested, in some aspects, there may be up to about 1,000,000 subjects tested, or up to about 500,000 subjects tested, or up to about 100,000 subjects tested. Such large numbers of test subjects offers good predictive assessment of skin sensitization for personal care product formulations.

At for example step 30, if all the ingredients do not meet the desired testing threshold thresholds, for example about ten or more formulations and about 2,000 or more subjects, then re-selection of the ingredients is recommended as the accuracy of the predictive assessment of skin sensitization may not be as accurate or precise as desired. As the predictive assessment of skin sensitization is used to as an alternative to physical formulation testing, use of conservative testing thresholds is desirable. In such as case where the desired testing thresholds are not met, one would return to step 10 in the flowchart of FIG. 1. Alternatively, the method of the present invention may be configured to only select possible ingredients meeting the desired testing threshold thresholds.

Ingredient skin sensitization values may be set such that each ingredient is at or below a skin sensitization criterion. Skin sensitization may be defined as a percentage of allergic responses for a group of subjects, such as:

$$( \sum subjects with confirmed allergic response \div \sum subjects completed patch test ) * ( 100 )$$

Frequency for adverse reactions typically used may be described as follows:
Very common, such as greater than or equal (≥) to about 10%;
Common, such as ≥ about 1% and less than (<) about 10%;
Uncommon, such as ≥ about 0.1% and < about 1%;
Rare, such as ≥ about 0.01% and < about 0.1%; and
Very rare, such < about 0.01%.
   Such characterizations of frequencies are non-limiting.

Desirably, the ingredient sensitization threshold or criteria at step 20 is set as a reasonably low value of adverse reactions, such as less than or equal (≤) about 0.2% for each ingredient. This threshold is non-limiting, and other thresholds may be used. For example, thresholds of ≤0.1%, ≤0.3%, ≤0.4%, ≤0.5%, and the like may suitably be used. In such as case where the desired ingredient sensitization threshold is not met, one would return to step 10 in the flowchart of FIG. 1. Alternatively, the method of the present invention may be configured to only select possible ingredients meeting the desired ingredient sensitization threshold.

At step 40, setting a further conservative ingredient sensitization threshold is performed by intentionally using an ingredient 95% upper confidence limit for the individual skin sensitization value for each ingredient. This 95% upper confidence limit or sensitization reaction score is obtained by using the total number of subjects tested (n) and total number of subjects with confirmed allergic reactions (a). An upper 95% confidence limit is obtained using the Clopper-Pearson exact confidence interval approach. This upper confidence limit applies to the population of all possible subjects treated in the exaggerated HRIPT patch testing conditions. Thus, numerically the upper 95% confidence limit value is greater than the mean value, thereby providing additional conservative mythology.

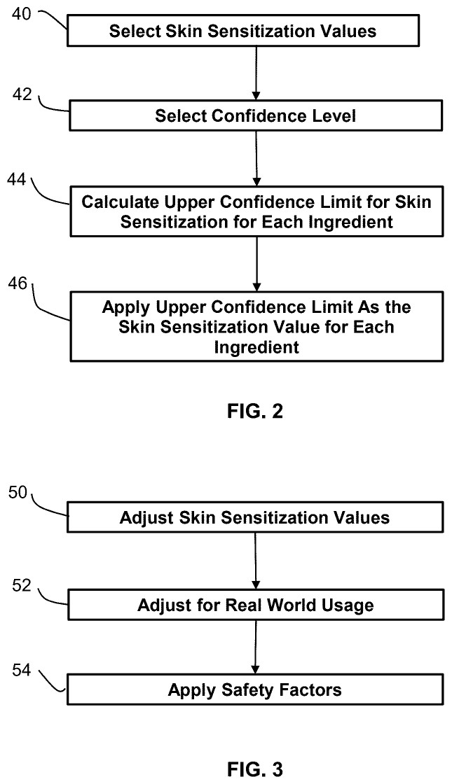

The step of selecting skin sensitization values at set 40 is further detailed in FIG. 2. At step 42, the confidence level is set at, for example 95%. If desired, other confidence levels may be selected from about 90% to about 99%. At step 44, the upper confidence limit for skin sensitization for each ingredient is determined as described above. At step 46, this upper confidence limit is applied as the skin sensitization value for each ingredient.

Returning to FIG. 1, the upper confidence limit for skin sensitization for each ingredient is further adjusted at step 50 to apply this upper confidence limit to an actual real-world consumer use of ingredients in a finished product adjustments.

Under typical conditions of exposure, the dose per unit area of a chemical may have an over-riding impact on the effectiveness of sensitization. As such, the methodology prediction model of the present invention uses a dose density adjustment based on real-life consumer use application for ingredients and finished product formulation. This approach conservatively allows the estimated probability of an event in real-world use of ingredients and finished product formulation matrix and improve the formulations safety design. An exemplary listing of product dose densities is described below in Table 1.

TABLE 1

| Product Category | Product Use Profiles | Use per Day (g) | Surface Area (cm²) |
|---|---|---|---|
| Body | Body Sunscreen | 18 | 17500 |
| Body | Body Moisturizer | 7.82 | 15670 |
| Body | Wash/Shower gel | 18.67 | 17500 |
| Face | Face Moisturizer | 1.54 | 565 |
| Face | Cleanser/Wash | 1.2 | 565 |
| Body - NA Habits | Sunscreen (6 month) | 4.07 | 3500 |
| Body - NA Habits | Moisturizer (newborn) | 1.57 | 2000 |
| Body - NA Habits | Wash (newborn) | 2.7 | 2000 |
| Body - NA Habits | Baby Wipe | 2.18 | 3500 |
| Hair/Scalp | Hair Styling products | 4 | 1010 |
| Hand | Hand sanitizer | 2.28 | 860 |
| Face | Make-up Removers | 5 | 565 |
| Eye | Eye shadow/make-up | 0.02 | 24 |
| Eye | Eyeliner | 0.01 | 3.2 |
| Eye | Mascara | 0.03 | 1.6 |
| Eye | Eye make-up remover | 0.5 | 50 |
| Face | Facial make-up (cream base) | 0.8 | 565 |
| Face | Skin whitening cream | 5 | 565 |
| Face | Liquid foundation | 0.51 | 565 |
| Face | Men's Aftershave | 1.2 | 305 |
| Foot | Foot Cream (Antiperspirant) | 2.4 | 1170 |
| Foot | Foot Cream (Antifungal) | 0.2 | 100 |
| Hand | Hand Cream | 2.16 | 860 |
| Body - NA Habits | Body Moisturizer (6 month) | 3.49 | 3500 |
| Body - NA Habits | Baby powder (6 month) | 4.15 | 3500 |
| Body - NA Habits | Nappy Cream (newborn) - Cosmetic | 1.32 | 2000 |
| Body - NA Habits | Baby powder (newborn) | 1.89 | 2000 |
| Hair/Scalp | Shampoo | 10.46 | 1440 |
| Hair/Scalp | Hair Conditioner | 3.92 | 1440 |
| Hair - NA Habits | Shampoo/Conditioner (6 month) | 4.15 | 3500 |
| Hair - NA Habits | Shampoo/Conditioner (newborn) | 1.87 | 2000 |
| Deodorant | Spray (ethanol-based) | 1.43 | 200 |
| Deodorant | Spray (not ethanol-based) | 0.69 | 200 |
| Deodorant | Stick/Roller (non-spray) | 1.5 | 200 |
| Face | Facial Scrub/Peeling gel (Normal) | 0.22 | 565 |
| Face | Facial Scrub (Daily) | 0.8 | 565 |
| Face | Men's Shaving cream/soap/foam | 2 | 305 |
| Hand | Hand Wash | 20 | 860 |
| Body - NA Habits | Body Wash (6 month) | 5.98 | 3500 |
| Body | Body Moisturizer (Compromised Skin) | 7.82 | 15670 |
| Face | Face Moisturizer (Compromised Skin) | 1.54 | 565 |
| Face | Facial Cleanser/Wash (Compromised Skin) | 1.2 | 565 |
| Body - NA Habits | Diaper Rash Cream (newborn) - OTC | 12 | 200 |
| Body - NA Habits | Baby Wipe | 2.18 | 3500 |
| Body - NA Habits | Baby Eczema | 3.5 | 3500 |
| Body - NA Habits | Nappy Cream (newborn) - Cosmetic | 1.32 | 200 |
| Face | Facial Scrub/Peeling gel (Normal) | 0.22 | 565 |
| Face | Facial Scrub (Daily) | 0.8 | 565 |
| Face | Men's Aftershave | 1.2 | 305 |
| Face | Acne Spot Treatment | 0.46 | 169.5 |

These values are non-limiting and may change over time to, for example, reflect changing consumer usages.

An adjustment factor for real-world expected use or dosage density is based on the use per day in grams divided by the application surface area in cm². Higher dosage weights or grams result in higher adjustment factors, while higher application areas in cm² result in lower adjustment factors. For example, a body sunscreen would have an adjustment factor of 18/17500 or about 0.00103, and antifungal foot cream would have an adjustment factor of 0.2/100 or about 0.002. Such adjustment factors are applied to the upper confidence limit for skin sensitization for each ingredient at step 52 in FIG. 3.

Continuing with FIG. 3, the dosage density adjusted upper confidence limits for skin sensitization may be further adjusted with safety factors at step 54 in FIG. 3.

Adjustments of safety factors is appropriate to conservatively include formulation matrix skin effect when predicting the probability for a real-life event extrapolated from the sum of the ingredient empirical patch data. The safety factor adjustments are applied based possible on (a) impairment to skin barrier function, (b) based on product delivery effecting skin occlusion and/or based on occlusive nature application site, (c) based potential for pre-conditioned skin having potential for activated inflammatory response, and/or combinations thereof. The present invention is not limited to the use of these three safety factors, and other and/or additional safety factors may suitably be used.

A safety factor (e.g., “safety factor a”) for barrier impacting ingredient inclusion or impairment to skin barrier function (e.g., alcohol-drying effect, surfactants, glycols, acids-disruptive, penetration enhancers) may be applied. Presence and level of ingredients (e.g., barrier disruptor ingredients such as Benzoyl Peroxide (anti-acne) and Retinol (anti-aging), among others) known or suspected to potentially be irritating in the final formulation with potential for an onset of inflammatory cascade may increase skin sensitization potential for the finished product for real-life consumer use. A safety factor of about 5 may be used. The value of 5 is non-limiting any other suitable safety values, such as 2, 3, 4, 6, 7, and 8 may be used. The safety factor may be applied across each ingredient or applied to select ingredients by multiplying the dosage density adjusted upper confidence limits for skin sensitization by the safety factor. If a safety factor is not applied, then the dosage density adjusted upper confidence limits for skin sensitization is multiplied by a safety factor of 1.

Another safety factor (e.g., “safety factor b”) may be based on a system likely to enhance delivery or a skin delivery permeation system effect. For example, occlusion of skin increases the hydration of stratum corneum, skin temperature, microbial count, pH, and dermal irritation and therefore may influence the penetration of the personal care product. As such it is important to note that under real-world consumer use scenarios leading to enhanced permeation whether it is as a result of an enhanced product delivery (occlusive masks, polymeric systems, nappy/diaper) or occlusive nature of application site such as underarm application or highly follicular areas, an additional safety factor is incorporated to maintain a conservative upper limit 95% confidence level for real-world consumer exposure of these occlusive scenarios based on the physicochemical property of the constituents. Other factors influencing this safety factor may include the polymeric system, an oil/water emulsion system, masks/clays with occlusivity system, and the like. A safety factor of about 5 may be used. The value of 5 is non-limiting any other suitable safety values such as 2, 3, 4, 6, 7, and 8 may be used. The safety factor may be applied across each ingredient or applied to select ingredients by multiplying the dosage density adjusted upper confidence limits for skin sensitization by the safety factor. If a safety factor is not applied, then the dosage density adjusted upper confidence limits for skin sensitization is multiplied by a safety factor of 1.

A further safety factor (e.g., “safety factor c”) may be based on primed immune skin condition. Potential activated inflammatory cascade, for example in the case of mechanical insult abraded shaved skin or sun burned skin, may inverse skin sensitization potential. Inflammation may be influenced directly by a chemical or mechanical trauma which may increase keratinocyte activity, and potentially increase the effect of primary skin irritation mediated sensitization response. Conservatively a skin sensitization assessment a safety factor may be applied for real-world consumer use upper limit confidence level extrapolation even though the inflammatory response is unrelated to the product application. A safety factor of about 2 may be used. The value of 2 is non-limiting any other suitable safety values, such as 3 or 4 may be used. The safety factor may be applied across each ingredient or applied to select ingredients by multiplying the dosage density adjusted upper confidence limits for skin sensitization by the safety factor. If a safety factor is not applied, then the dosage density adjusted upper confidence limits for skin sensitization is multiplied by a safety factor of 1.

Numerical values to estimate probability of skin sensitization may be generated based on application of safety factors that emulate real-world conditions of use. Several considerations may result in application of safety factors to sets of combined ingredients that make up a proposed or final product formulation, as follows:
1) When one or more of the ingredients in the ingredient sets are classified as barrier disruptors (“BD”) (examples: alcohol-drying effect, glycols, acids, and penetration enhancers), then a safety factor of, for example, 5× may be applied to each ingredient of the formula ingredient set.
2) Another safety factor of, for example, 5× may be applied if the formula delivery (“DE”) type facilitates increased bio-permeability (such as the case of serum polymeric systems, or occlusive delivery, such as masks, or inclusion of high level of occlusive ingredients).
3) A safety factor of, for example, 2× may be applied in case of primed immune skin end use (“PEU”) conditions, for example after-sun use, acne use.

Table 2 below illustrates numerical values for formula ingredient sets with the achieved numerical values based on respective safety factor applications. It is noted that as more safety factors are applied, then the greater the numerical value for an ingredient set relative to the number of ingredients. For example, a formula with 17 ingredients and a safety factor adjustment for PEU (2×) results in a value of 0.0007%, whereas a formula with 29 ingredients and a safety factor application for both PEU (2×) and BD (5×) would have a 0.062% value. Alternatively, a formula with 13 ingredients and a safety factor adjustment of 1× would have a 0.0008% value.

TABLE 2

| | Formula | Number of | Safety Factory | Adjusted UCL(p)- |
|---|---|---|---|---|
| | Ingredient | Ingredients | Adjusters (overall | (Target Sensiti- |
| Product Type | Sets | in the Set | combined factor) | zation ≤0.1%) |
| After sun (primed | F-1 | 17 | PEU (2x) | 0.0007 |
| immune skin end use) | | | | |
| Face moisturizer | F-2 | 12 | BD & DE (25x) | 0.046 |
| (barrier disruptor | | | | |
| ingredient - | | | | |
| serum delivery) | | | | |
| Face moisturizer | F-3 | 11 | BD & DE (25x) | 0.0441 |
| (barrier disruptor | | | | |
| ingredient - | | | | |
| serum delivery) | | | | |
| Face moisturizer | F-4 | 29 | BD (5x) | 0.027 |
| (barrier disrupter | | | | |
| ingredient) | | | | |
| Spot treatment | F-5 | 29 | PEU & BD (10x) | 0.062 |
| anti-acne | | | | |
| (barrier disrupter | | | | |
| ingredient- | | | | |
| Primed End Use) | | | | |
| Baby wash | F-6 | 13 | No Adjustors | 0.0008 |
| (rinse off) | | | Applied (1x) | |
| Face mask acne | F-7 | 15 | PEU & BD & DE | 0.422 (re- |
| (barrier disruptor | | | (50x) | formulation |
| ingredient- | | | | may be |
| occlusive delivery- | | | | recommended) |
| primed end use) | | | | |

Real-world data product surveillance for cutaneous events may be used to support a trend for the application of above safety factors. For example, a product that falls under DE & BD would have a higher value for observed cutaneous events as compared to one that would include only DE, or BD or none.

Data in the table below captures the real-world data for product delivery enhancer systems (DE: yes, no) which may also contain barrier disruptor ingredients (BD: yes, no). For example, a product with DE & BD may have a percent observed value of 0.056%, whereas a product with only BD may have a value of 0.027%.

TABLE 3

| | |
|---|---|
| Delivery | Ingredient Barrier Disruptor (BD) |

| Enhancer (DE) | No | yes |
|---|---|---|
| No | 0.013% (0.011%) | 0.027% (0.029%) |
| Yes | 0.019% (0.020%) | 0.056% (0.051%) |

The value for the percent of users with cutaneous observed events may be calculated based on the observed number of subjects with events/number units sold=% observed with cutaneous events. The predicted probability may be calculated based on logistic regression model with terms for Delivery Enhancer and Barrier Disrupter and these values are shown in parentheses.

In summary, the dosage density adjusted upper confidence limits for skin sensitization for select or all of the ingredients may be multiplied by “safety factor a” and by “safety factor b” and by “safety factor c” to obtain an adjusted skin sensitization values.

Returning to FIG. 1, a target product sensitization value or threshold is set at step 60. Desirably, the target product sensitization at ≤ about 0.1%. This value is non-limiting and other target sensitization values may be used. Desirably, the target product sensitization value (e.g., ≤about 0.1%) is less than the ingredient sensitization threshold value or the initial sensitization target (e.g., ≤ about 0.2%).

The adjusted skin sensitization values for each ingredient from step 50 are summed in step 70 to define a total sensitization or calculated total sensitization for proposed personal care product formulation. No further adjustments are normally needed here, but if some for reason additional adjustments are warranted, then they may be suitably applied at this step 70.

At step 80, if the calculated total sensitization for proposed personal care product formulation is greater than the target product sensitization, then product re-formulation may be warranted. If the calculated total sensitization for proposed personal care product formulation is less than the target product sensitization, then at step 90 the proposed personal care product formulation is capable of application to skin of a user without, for example, further physical product testing or possibly just minimal product testing such testing with reduced number of subjects.

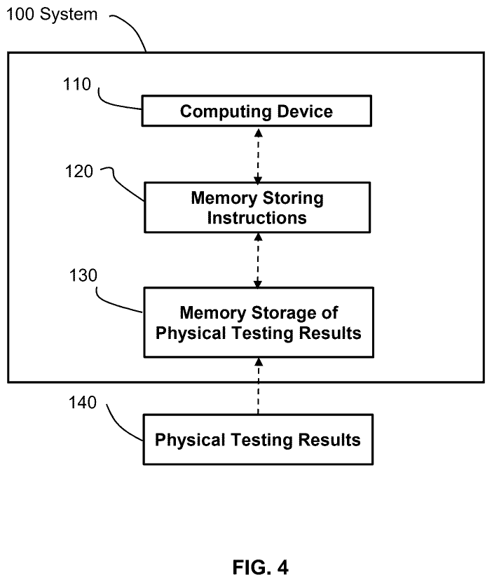

FIG. 4 schematically depicts a system 100 to predictively assess skin sensitization of a personal care product formulation including a plurality of ingredients. The system 100 includes memory storage of physical testing results 130. The physical resting results 140 included 1,274 formulations tested across 203,640 subjects in HRIPT tests. The formulations reused 1,226 common constituents with 99.98% of the tested subjects (203,602 out of 203,640 subjects) exhibited zero confirmed allergic response. About 0.02% of the tested subjects (38 out of 203,640 subjects) exhibited some allergic response. These values are non-limiting and are offered to illustrate population and test sizes. For example, as additional physical results become available, they may be added into the memory storage of physical testing results 130.

Memory storing instructions 120 that, when executed by the computing device 110, cause the computing device to perform operations including, but not limited to:
calculating an upper confidence limit at a desired confidence level from multiple tests for skin sensitization of each of the ingredients in the personal care product formulation in order to define calculated skin sensitization values for respective ingredients;
applying at least one safety factor to the calculated skin sensitization values for the ingredients to define adjusted skin sensitization values for the respective ingredients;
summing the adjusted skin sensitization values of the ingredients to define a total sensitization of the personal care product formulation; and
comparing the total sensitization to a target product sensitization for the personal care product formulation.

The memory storing instructions 120 may include any of the calculations, data manipulations, logic decisions, sequence instructions described herein.

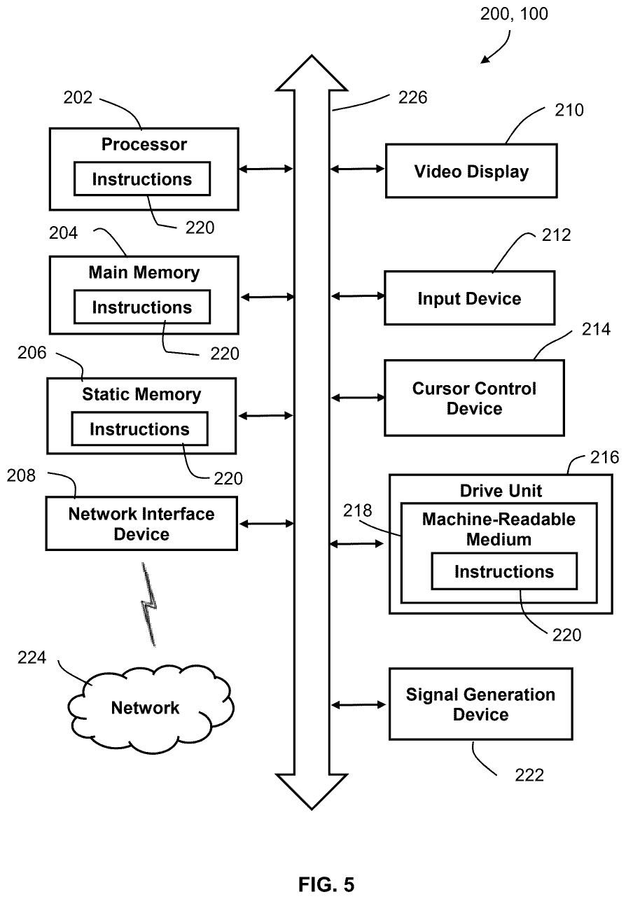

FIG. 5 is a block diagram of an illustrative embodiment of a general computer system 200 which further details of the system 100 of FIG. 4. The computer system 200 can include a set of instructions that can be executed to cause the computer system 200 to perform any one or more of the methods or computer-based functions disclosed herein, including in FIGS. 1-4. The computer system 200, or any portion thereof, may operate as a standalone device or may be connected, e.g., using a network or other connection, to other computer systems or peripheral devices. For example, the computer system 200 may be a computing device 110, and may further be connected to other systems and devices, such as content providers, via a network.

The computer system 200 may also be implemented as or incorporated into various devices, such as a personal computer (PC), a tablet PC, a personal digital assistant (PDA), a computing device or mobile device (e.g., smartphone), a palmtop computer, a laptop computer, a desktop computer, a communications device, a control system, a web appliance, or any other machine capable of executing a set of instructions (sequentially or otherwise) that specify actions to be taken by that machine. Further, while a single computer system 200 is illustrated, the term “system” shall also be taken to include any collection of systems or sub-systems that individually or jointly execute a set, or multiple sets, of instructions to perform one or more computer functions.

As illustrated in FIG. 5, the computer system 200 may include a processor 202, e.g., a central processing unit (CPU), a graphics-processing unit (GPU), or both. Moreover, the computer system 200 may include a main memory 204 and a static memory 206 that can communicate with each other via a bus 226. As shown, the computer system 200 may further include a video display unit 210, such as a liquid crystal display (LCD), an organic light emitting diode (OLED), a flat panel display, a solid state display, or a cathode ray tube (CRT). Additionally, the computer system 200 may include an input device 212, such as a keyboard, and a cursor control device 214, such as a mouse. The computer system 200 can also include a disk drive (or solid state) unit 216, a signal generation device 222, such as a speaker or remote control, and a network interface device 208.

In a particular embodiment or aspect, as depicted in FIG. 5, the disk drive (or solid state) unit 216 may include a computer-readable medium 218 in which one or more sets of instructions 220, e.g., software, can be embedded. Further, the instructions 220 may embody one or more of the methods or logic as described herein. In a particular embodiment or aspect, the instructions 220 may reside completely, or at least partially, within the main memory 204, the static memory 206, and/or within the processor 202 during execution by the computer system 200. The main memory 204 and the processor 202 also may include computer-readable media.

Dedicated hardware implementations, such as application specific integrated circuits, programmable logic arrays and other hardware devices, can be constructed to implement one or more of the methods described herein. Applications that may include the apparatus and systems of various embodiments or aspects can broadly include a variety of electronic and computer systems. One or more embodiments or aspects described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The methods described herein may be implemented by software programs tangibly embodied in a processor-readable medium and may be executed by a processor. Further, in an exemplary, non-limited embodiment or aspect, implementations can include distributed processing, component/object distributed processing, and parallel processing. Alternatively, virtual computer system processing can be constructed to implement one or more of the methods or functionality as described herein.

It is also contemplated that a computer-readable medium includes instructions 220 or receives and executes instructions 220 responsive to a propagated signal, so that a device connected to a network 224 can communicate voice, video, or data over the network 224. Further, the instructions 220 may be transmitted or received over the network 224 via the network interface device 208.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, example embodiment or aspect, the computer-readable medium can include a solid-state memory, such as a memory card or other package, which houses one or more non-volatile read-only memories. Further, the computer-readable medium can be a random-access memory or other volatile re-writable memory. Additionally, the computer-readable medium can include a magneto-optical or optical medium, such as a disk or tapes or other storage device to capture carrier wave signals, such as a signal communicated over a transmission medium. A digital file attachment to an e-mail or other self-contained information archive or set of archives may be considered a distribution medium that is equivalent to a tangible storage medium. Accordingly, any one or more of a computer-readable medium or a distribution medium and other equivalents and successor media, in which data or instructions may be stored, are included herein.

The methods described herein may be implemented as one or more software programs running on a computer processor. Dedicated hardware implementations including, but not limited to, application specific integrated circuits, programmable logic arrays, and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

Software that implements the disclosed methods may optionally be stored on a tangible storage medium, such as: a magnetic medium, such as a disk or tape; a magneto-optical or optical medium, such as a disk; or a solid-state medium, such as a memory card or other package that houses one or more read-only (non-volatile) memories, random access memories, or other re-writable (volatile) memories. The software may also utilize a signal containing computer instructions. A digital file attachment to e-mail or other self-contained information archive or set of archives is considered a distribution medium equivalent to a tangible storage medium. Accordingly, a tangible storage medium or distribution medium as listed herein, and other equivalents and successor media, in which the software implementations herein may be stored, are included herein.

The features and advantages of the present invention are more fully shown by the following examples which are provided for purposes of illustration, and are not to be construed as limiting the invention in any way.

In the following Examples, various ingredients were used for evaluation purposes. The below Table identifies various ingredients that were evaluated in one or more of the Examples herein. These ingredients are only some of the ingredients that may be evaluated through the present invention and are not to be construed as limiting the invention in any way.

### Ingredients:

| INCI | Function |
|---|---|
| Benzoyl Peroxide | Anti-acne agent |
| Salicylic Acid | Anti-acne agent |
| Retinol | Anti-aging agent |
| Benzalkonium Chloride | Antimicrobial agent |
| Ascorbic Acid | Antioxidant |
| Ascorbyl Palmitate | Antioxidant |
| BHT | Antioxidant |
| Pentaerythrityl Tetra-di-t-butyl | Antioxidant |
| Hydroxyhydrocinnamate | |
| Tocopherol | Antioxidant |
| Tocopheryl Acetate | Antioxidant |
| Hamamelis Virginiana (Witch Hazel) Water | Astringent |
| Polyisobutene | Binder |
| Sodium Citrate | Buffering agent |
| Sodium Hydroxide | Buffering agent |
| Disodium EDTA | Chelating agent |
| Tetrasodium Glutamate Diacetate | Chelating agent |
| Menthol | Cooling agent |
| Caprylyl Glycol | Emollient |
| Dicaprylyl Carbonate | Emollient |
| Glyceryl Behenate | Emollient |
| Hydrogenated Palm Glycerides | Emollient |
| Isohexadecane | Emollient |
| Mineral Oil | Emollient |
| Pentaerythrityl Tetraethylhexanoate | Emollient |
| PPG-15 Stearyl Ether | Emollient |
| Triethyl Citrate | Emollient |
| Ceteareth-20 | Emulsifier |
| Cetearyl Alcohol | Emulsifier |
| Lecithin | Emulsifier |
| Polysorbate 20 | Emulsifier |
| Potassium Cetyl Phosphate | Emulsifier |
| Sodium Polyacrylate | Emulsifier |
| Stearyl Alcohol | Emulsifier |
| Hydroxyethylcellulose | Film Former |
| Polyacrylate-13 | Film Former |
| Fragrance | Fragrance |
| Butylene Glycol | Humectant |
| Glycerin | Humectant |
| p-Anisic Acid | Masking Agent |
| Kaolin | Occlusive Absorbent |
| Bentonite | Occlusive Absorbent |
| Titanium Dioxide | Opacifying agent |
| Citric Acid | pH adjuster |
| Benzyl Alcohol | Preservative |
| Chlorphenesin | Preservative |
| Ethylhexylglycerin | Preservative |
| Phenoxyethanol | Preservative |
| Sodium Benzoate | Preservative |
| Aloe Barbadensis Leaf Juice | Skin conditioner |
| Butyrospermum Parkii (Shea) Butter | Skin conditioner |
| C12-15 Alkyl Lactate | Skin conditioner |
| Cedrus Atlantica Bark Extract | Skin conditioner |
| Cellulose | Skin conditioner |
| Cetyl Lactate | Skin conditioner |
| Cinnamomum Zeylanicum Bark Extract | Skin conditioner |
| Cocamidopropyl PG-Dimonium Chloride | Skin conditioner |
| Phosphate | |
| Coco-Glucoside | Skin conditioner |
| Cyclopentasiloxane | Skin conditioner |
| Dimethicone | Skin conditioner |
| Dimethicone Crosspolymer | Skin conditioner |
| Glyceryl Oleate | Skin conditioner |
| Portulaca Oleracea Extract | Skin conditioner |
| PPG-2 Isoceteth-20 Acetate | Skin conditioner |
| Sarcosine | Skin conditioner |
| Sodium Hyaluronate | Skin conditioner |
| C13-14 Isoparaffin | Slip modifier |
| Propylene Glycol | Solubilizer |
| Capryloyl Glycine | Surfactant |
| Cocamidopropyl Betaine | Surfactant |
| Decyl Glucoside | Surfactant |
| PEG-80 Sorbitan Laurate | Surfactant |
| Sodium Methyl Cocoyl Taurate | surfactant |
| Alcohol | Vehicle |
| Water | Vehicle |
| Dehydroxanthan Gum | Viscosity controlling |
| | agent |
| Acrylates/C10-30 Alkyl Acrylate Crosspolymer | Viscosity Increasing |
| | agent |
| Carbomer | Viscosity increasing |
| | agent |
| Laureth-7 | Viscosity increasing |
| | agent |
| Sodium Acryloyldimethyltaurate/VP | Viscosity increasing |
| Crosspolymer | agent |
| Xanthan Gum | Viscosity increasing |
| | agent |

### EXAMPLE 1

An After Sun Lotion was formulated with the following ingredients as shown in Table 4 below:

TABLE 4

| Proposed After Sun, Lotion | Ingredient |
|---|---|
| Formula Ingredient Composition | No. |
| Skin conditioner | A1 |
| Emulsifier | A2 |
| Antioxidant | A3 |
| Skin conditioner | A4 |
| Antioxidant | A5 |
| Masking Agent | A6 |
| Emulsifier | A7 |
| Fragrance | A8 |
| Emollient | A9 |
| Emollient | A10 |
| Humectant | A11 |
| Preservative | A12 |
| Vehicle | A13 |
| Antioxidant | A14 |
| Emollient | A15 |
| pH adjuster | A16 |
| Viscosity increasing agent | A17 |

All of the ingredients were used in ten or more personal care products. The number of tested formulas containing the above ingredients varied from 20 to 1,176. All of the ingredients were tested on 2,000 or more subjects. The total number of subjects tested in formulas containing the above ingredient varied from 3,570 to 187,429.

The ingredient sensitization for each of the ingredients is shown in Table 5 below. The upper confidence limit (UCL(p)) at 95% confidence level was calculated for each ingredient. A dosage density factor 7.82/15670 (representing 7.82 g of daily dose over 15,670 cm² usage surface area from Table 1 above) was applied to the upper confidence limit for each ingredient. These values are shown below in Table 5 below.

TABLE 5

| Ingredient | Ingredient | Ingredient | Dosage Density Adjusted |
|---|---|---|---|
| No. | Sensitization (%) | UCL(p) | Ingredient Sensitization |
| A1 | 0.016 | 0.074 | 3.70E−05 |
| A2 | 0.000 | 0.084 | 4.19E−05 |
| A3 | 0.000 | 0.047 | 2.36E−05 |
| A4 | 0.005 | 0.022 | 1.11E−05 |
| A5 | 0.008 | 0.017 | 8.45E−06 |
| A6 | 0.010 | 0.048 | 2.37E−05 |
| A7 | 0.011 | 0.029 | 1.44E−05 |
| A8 | 0.014 | 0.020 | 9.94E−06 |
| A9 | 0.014 | 0.067 | 3.35E−05 |
| A10 | 0.016 | 0.028 | 1.42E−05 |
| A11 | 0.017 | 0.024 | 1.18E−05 |
| A12 | 0.017 | 0.025 | 1.25E−05 |
| A13 | 0.019 | 0.025 | 1.24E−05 |
| A14 | 0.021 | 0.037 | 1.85E−05 |
| A15 | 0.023 | 0.052 | 2.60E−05 |
| A16 | 0.024 | 0.034 | 1.67E−05 |
| A17 | 0.041 | 0.067 | 3.36E−05 |

As used herein the scientific notation of the format “x.xxE-yy” represents x.xx 10^{-yy} or x.xx*10^{-yy}. Thus, for example, 3.70E-05 represents 0.0000370.

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, as needed, for safety factors. In this example the (a) Applied Safety Factor: Delivery System was “1”; the (b) Applied Safety Factor: Ingredients Modulating Skin Barrier to Enhance Bio-delivery was “1”; and the (c) Applied Safety Factor: Primed Immune Skin Condition Intended Use was “2”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category was not applicable. The safety adjusted sensitization values are shown below.

TABLE 6

| | | (b) Applied Safety | |
|---|---|---|---|
| | | Factor (“1”): | (c) Applied Safety |
| | | Ingredients | Factor (“2”): |
| Ingre- | (a) Applied Safety | Modulating Skin | Primed Immune |
| dient | Factor (“1”): | Barrier to Enhance | Skin Condition |
| No. | Delivery System | Bio-delivery | Intended Use |
| A1 | 3.70E−05 | 3.70E−05 | 7.41E−05 |
| A2 | 4.19E−05 | 4.19E−05 | 8.37E−05 |
| A3 | 2.36E−05 | 2.36E−05 | 4.72E−05 |
| A4 | 1.11E−05 | 1.11E−05 | 2.22E−05 |
| A5 | 8.45E−06 | 8.45E−06 | 1.69E−05 |
| A6 | 2.37E−05 | 2.37E−05 | 4.75E−05 |
| A7 | 1.44E−05 | 1.44E−05 | 2.88E−05 |
| A8 | 9.94E−06 | 9.94E−06 | 1.99E−05 |
| A9 | 3.35E−05 | 3.35E−05 | 6.70E−05 |
| A10 | 1.42E−05 | 1.42E−05 | 2.83E−05 |
| A11 | 1.18E−05 | 1.18E−05 | 2.37E−05 |
| A12 | 1.25E−05 | 1.25E−05 | 2.49E−05 |
| A13 | 1.24E−05 | 1.24E−05 | 2.47E−05 |
| A14 | 1.85E−05 | 1.85E−05 | 3.71E−05 |
| A15 | 2.60E−05 | 2.60E−05 | 5.19E−05 |
| A16 | 1.67E−05 | 1.67E−05 | 3.35E−05 |
| A17 | 3.36E−05 | 3.36E−05 | 6.72E−05 |

The order of the category of the applied safety factors was not critical. Indeed, the category of the applied safety factors may be provided in any desirable order.

The safety factor adjusted sensitization values were then summed, as shown in Table 7 below.

TABLE 7

| | Ingredient Real-World Application |
|---|---|
| Ingredient | Sensitization Values Adjusted for |
| No. | Intended Application And Safety Factors |
| A1 | 7.41E−05 |
| A2 | 8.37E−05 |
| A3 | 4.72E−05 |
| A4 | 2.22E−05 |
| A5 | 1.69E−05 |
| A6 | 4.75E−05 |
| A7 | 2.88E−05 |
| A8 | 1.99E−05 |
| A9 | 6.70E−05 |
| A10 | 2.83E−05 |
| A11 | 2.37E−05 |
| A12 | 2.49E−05 |
| A13 | 2.47E−05 |
| A14 | 3.71E−05 |
| A15 | 5.19E−05 |
| A16 | 3.35E−05 |
| A17 | 6.72E−05 |
| Total Sum: | 0.00070 |

As the calculated sensitization value of 0.00070 for the proposed formulation was less than a typical product sensitization value or threshold of 0.1, the proposed formulation is capable of application to the skin of users without or with just minimal testing as it is correlated with real-world data.

### EXAMPLE 2

A Face Moisturizer in the form of a serum and having Anti-aging ingredient was formulated with the following ingredients as shown in Table 8 below:

TABLE 8

| Proposed Face Moisturizer (Serum - | |
|---|---|
| Anti-aging ingredient) Formula | Ingredient |
| Ingredient Composition | No. |
| Antioxidant | B1 |
| Emulsifier | B2 |
| Emollient | B3 |
| Antioxidant | B4 |
| Emollient | B5 |
| Fragrance | B6 |
| Emollient | B7 |
| Antioxidant | B8 |
| Antioxidant | B9 |
| Emollient | B10 |
| Emulsifier | B11 |
| Anti-aging | B12 |

All of the ingredients were used in ten or more personal care products. The number of tested formulas containing the above ingredients varied from 23 to 958. All of the ingredients were tested on 2,000 or more subjects. The total number of subjects tested in formulas containing the above ingredient varied from 3,448 to 151,896.

The ingredient sensitization for each of the ingredients is shown in Table 9 below. The upper confidence limit (UCL(p)) at 95% confidence level was calculated for each ingredient. A dosage density factor 1.54/565 (representing 1.54 g of daily dose over 565 cm² usage surface area from Table 1 above) was applied to the upper confidence limit for each ingredient. These values are shown below in Table 9 below.

TABLE 9

| Ingredient | Ingredient | Ingredient | Dosage Density Adjusted |
|---|---|---|---|
| No. | Sensitization (%) | UCL(p) | Ingredient Sensitization |
| B1 | 0.000 | 0.039 | 1.06E−04 |
| B2 | 0.000 | 0.089 | 2.43E−04 |
| B3 | 0.0155 | 0.040 | 1.10E−04 |
| B4 | 0.008 | 0.017 | 4.61E−05 |
| B5 | 0.010 | 0.048 | 1.29E−04 |
| B6 | 0.014 | 0.020 | 5.43E−05 |
| B7 | 0.014 | 0.066 | 1.79E−04 |
| B8 | 0.015 | 0.040 | 1.09E−04 |
| B9 | 0.017 | 0.035 | 9.55E−05 |
| B10 | 0.017 | 0.081 | 2.22E−04 |
| B11 | 0.028 | 0.052 | 1.41E−04 |
| B12 | 0.051 | 0.159 | 4.34E−04 |

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, if needed, for safety factors. In this example the (a) Applied Safety Factor: Delivery System was “5”; the (b) Applied Safety Factor: Ingredients Modulating Skin Barrier to Enhance Bio-delivery was “5”; and the (c) Applied Safety Factor: Primed Immune Skin Condition Intended Use was “1”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category is not applicable. The safety adjusted sensitization values are shown below.

TABLE 10

| | | (b) Applied Safety | |
|---|---|---|---|
| | | Factor (“5”): | (c) Applied Safety |
| | | Ingredients | Factor (“1”): |
| Ingre- | (a) Applied Safety | Modulating Skin | Primed Immune |
| dient | Factor (“5”): | Barrier to Enhance | Skin Condition |
| No. | Delivery System | Bio-delivery | Intended Use |
| B1 | 5.30E−04 | 2.65E−03 | 2.65E−03 |
| B2 | 1.22E−03 | 6.08E−03 | 6.08E−03 |
| B3 | 5.50E−04 | 2.75E−03 | 2.75E−03 |
| B4 | 2.31E−04 | 1.15E−03 | 1.15E−03 |
| B5 | 6.47E−04 | 3.24E−03 | 3.24E−03 |
| B6 | 2.71E−04 | 1.36E−03 | 1.36E−03 |
| B7 | 8.94E−04 | 4.47E−03 | 4.47E−03 |
| B8 | 5.43E−04 | 2.71E−03 | 2.71E−03 |
| B9 | 4.78E−04 | 2.39E−03 | 2.39E−03 |
| B10 | 1.11E−03 | 5.54E−03 | 5.54E−03 |
| B11 | 7.07E−04 | 3.53E−03 | 3.53E−03 |
| B12 | 2.17E−03 | 1.08E−02 | 1.08E−02 |

The order of the category of the applied safety factors was not critical. Indeed, the category of the applied safety factors may be provided in any desirable order.

The safety factor adjusted sensitization values were then summed, as shown in Table 11 below.

TABLE 11

| | Ingredient Real-World Application |
|---|---|
| Ingredient | Sensitization Values Adjusted for |
| No. | Intended Application And Safety Factors |
| B1 | 2.65E−03 |
| B2 | 6.08E−03 |
| B3 | 2.75E−03 |
| B4 | 1.15E−03 |
| B5 | 3.24E−03 |
| B6 | 1.36E−03 |
| B7 | 4.47E−03 |
| B8 | 2.71E−03 |
| B9 | 2.39E−03 |
| B10 | 5.54E−03 |
| B11 | 3.53E−03 |
| B12 | 1.08E−02 |
| Total Sum: | 0.0467 |

As the calculated sensitization value of 0.0467 for the proposed formulation was less than a typical product sensitization value or threshold of 0.1, the proposed formulation is capable of application to the skin of users without or with just minimal testing as it is correlated with real-world data.

### EXAMPLE 3

A Face Moisturizer having anti-aging ingredient was formulated with the following ingredients as shown in Table 12 below:

TABLE 12

| Face Moisturizer (Anti-aging ingredient) | Ingredient |
|---|---|
| Formula Ingredient Composition | No. |
| Emulsifier | C1 |
| Emollient | C2 |
| Antioxidant | C3 |
| Emollient | C4 |
| Fragrance | C5 |
| Emollient | C6 |
| Antioxidant | C7 |
| Antioxidant | C8 |
| Emollient | C9 |
| Emulsifier | C10 |
| Anti-aging | C11 |

All of the ingredients were used in ten or more personal care products. The number of tested formulas containing the above ingredients varied from 23 to 958. All of the ingredients were tested on 2,000 or more subjects. The total number of subjects tested in formulas containing the above ingredient varied from 3,448 to 151,895.

The ingredient sensitization for each of the ingredients is shown in Table 13 below. The upper confidence limit (UCL(p)) at 95% confidence level was calculated for each ingredient. A dosage density factor 1.54/565 (representing 1.54 g of daily dose over 565 cm² usage surface area from Table 1 above) was applied to the upper confidence limit for each ingredient. These values are shown below in Table 13 below.

TABLE 13

| Ingredient | Ingredient | Ingredient | Dosage Density Adjusted |
|---|---|---|---|
| No. | Sensitization (%) | UCL(p) | Ingredient Sensitization |
| C1 | 0.000 | 0.089 | 2.43E−04 |
| C2 | 0.015 | 0.040 | 1.10E−04 |
| C3 | 0.008 | 0.017 | 4.61E−05 |
| C4 | 0.010 | 0.048 | 1.29E−04 |
| C5 | 0.014 | 0.020 | 5.43E−05 |
| C6 | 0.014 | 0.066 | 1.79E−04 |
| C7 | 0.015 | 0.040 | 1.09E−04 |
| C8 | 0.017 | 0.035 | 9.55E−05 |
| C9 | 0.017 | 0.081 | 2.22E−04 |
| C10 | 0.028 | 0.052 | 1.41E−04 |
| C11 | 0.051 | 0.159 | 4.34E−04 |

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, if needed, for safety factors. In this example the (a) Applied Safety Factor: Delivery System was “5”; the (b) Applied Safety Factor: Ingredients Modulating Skin Barrier to Enhance Bio-delivery was “5”; and the (c) Applied Safety Factor: Primed Immune Skin Condition Intended Use was “1”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category is not applicable. The safety adjusted sensitization values are shown below.

TABLE 14

| | | (b) Applied Safety | |
|---|---|---|---|
| | | Factor (“5”): | (c) Applied Safety |
| | | Ingredients | Factor (“1”): |
| Ingre- | (a) Applied Safety | Modulating Skin | Primed Immune |
| dient | Factor (“5”): | Barrier to Enhance | Skin Condition |
| No. | Delivery System | Bio-delivery | Intended Use |
| C1 | 1.22E−03 | 6.08E−03 | 6.08E−03 |
| C2 | 5.50E−04 | 2.75E−03 | 2.75E−03 |
| C3 | 2.31E−04 | 1.15E−03 | 1.15E−03 |
| C4 | 6.47E−04 | 3.24E−03 | 3.24E−03 |
| C5 | 2.71E−04 | 1.36E−03 | 1.36E−03 |
| C6 | 8.94E−04 | 4.47E−03 | 4.47E−03 |
| C7 | 5.43E−04 | 2.71E−03 | 2.71E−03 |
| C8 | 4.78E−04 | 2.39E−03 | 2.39E−03 |
| C9 | 1.11E−03 | 5.54E−03 | 5.54E−03 |
| C10 | 7.07E−04 | 3.53E−03 | 3.53E−03 |
| C11 | 2.17E−03 | 1.08E−02 | 1.08E−02 |

The order of the category of the applied safety factors was not critical. Indeed, the category of the applied safety factors may be provided in any desirable order.

The safety factor adjusted sensitization values were then summed, as shown in Table 15 below.

TABLE 15

| | Ingredient Real-World Application |
|---|---|
| Ingredient | Sensitization Values Adjusted for |
| No. | Intended Application And Safety Factors |
| C1 | 6.08E−03 |
| C2 | 2.75E−03 |
| C3 | 1.15E−03 |
| C4 | 3.24E−03 |
| C5 | 1.36E−03 |
| C6 | 4.47E−03 |
| C7 | 2.71E−03 |
| C8 | 2.39E−03 |
| C9 | 5.54E−03 |
| C10 | 3.53E−03 |
| C11 | 1.08E−02 |
| Total Sum: | 0.0441 |

As the calculated sensitization value of 0.0441 for the proposed formulation was less than a typical product sensitization value or threshold of 0.1, the proposed formulation is capable of application to the skin of users without or with just minimal testing as it is correlated with real-world data.

### EXAMPLE 4

A Face Moisturizer having anti-aging ingredient was formulated with the following ingredients as shown in Table 16 below:

TABLE 16

| Proposed Face Moisturizer | |
|---|---|
| (Anti-aging ingredient) | Ingredient |
| Formula Ingredient Composition | No. |
| Emollient | D1 |
| Viscosity increasing agent | D2 |
| Emollient | D3 |
| Skin Conditioner | D4 |
| Emollient | D5 |
| Emulsifier | D6 |
| Humectant | D7 |
| Antioxidant | D8 |
| Fragrance | D9 |
| Skin conditioner | D10 |
| Antioxidant | D11 |
| Humectant | D12 |
| Emollient | D13 |
| Preservative | D14 |
| Skin conditioner | D15 |
| Vehicle | D16 |
| Skin conditioner | D17 |
| Emulsifier | D18 |
| pH adjuster | D19 |
| Chelating agent | D20 |
| Slip modifier | D21 |
| Viscosity increasing agent | D22 |
| Emulsifier | D23 |
| Emulsifier | D24 |
| Preservative | D25 |
| Skin conditioner | D26 |
| Skin conditioner | D27 |
| Emulsifier | D28 |
| Anti-aging agent | D29 |

All of the ingredients were used in ten or more personal care products. The number of tested formulas containing the above ingredients varied from 12 to 1,176. All of the ingredients were tested on 2,000 or more subjects. The total number of subjects tested in formulas containing the above ingredient varied from 2,000 to 187,429.

The ingredient sensitization for each of the ingredients is shown in Table 17 below. The upper confidence limit (UCL(p)) at 95% confidence level was calculated for each ingredient. A dosage density factor 1.54/565 (representing 1.54 g of daily dose over 565 cm² usage surface area from Table 1 above) was applied to the upper confidence limit for each ingredient. These values are shown below in Table 17 below.

TABLE 17

| Ingredient | Ingredient | Ingredient | Dosage Density Adjusted |
|---|---|---|---|
| No. | Sensitization (%) | UCL(p) | Ingredient Sensitization |
| D1 | 0.000 | 0.121 | 3.30E−04 |
| D2 | 0.008 | 0.040 | 1.09E−04 |
| D3 | 0.000 | 0.156 | 4.26E−04 |
| D4 | 0.006 | 0.019 | 5.28E−05 |
| D5 | 0.010 | 0.048 | 1.30E−04 |
| D6 | 0.010 | 0.048 | 1.31E−04 |
| D7 | 0.012 | 0.023 | 6.26E−05 |
| D8 | 0.012 | 0.058 | 1.58E−04 |
| D9 | 0.014 | 0.020 | 5.43E−05 |
| D10 | 0.016 | 0.028 | 7.73E−05 |
| D11 | 0.017 | 0.035 | 9.55E−05 |
| D12 | 0.017 | 0.024 | 6.47E−05 |
| D13 | 0.017 | 0.081 | 2.22E−04 |
| D14 | 0.017 | 0.025 | 6.81E−05 |
| D15 | 0.017 | 0.027 | 7.39E−05 |
| D16 | 0.019 | 0.025 | 6.75E−05 |
| D17 | 0.020 | 0.063 | 1.70E−04 |
| D18 | 0.022 | 0.040 | 1.10E−04 |
| D19 | 0.024 | 0.034 | 9.15E−05 |
| D20 | 0.024 | 0.036 | 9.74E−05 |
| D21 | 0.025 | 0.064 | 1.73E−04 |
| D22 | 0.025 | 0.064 | 1.73E−04 |
| D23 | 0.028 | 0.052 | 1.41E−04 |
| D24 | 0.028 | 0.053 | 1.45E−04 |
| D25 | 0.033 | 0.060 | 1.63E−04 |
| D26 | 0.043 | 0.099 | 2.70E−04 |
| D27 | 0.047 | 0.222 | 6.06E−04 |
| D28 | 0.047 | 0.224 | 6.09E−04 |
| D29 | 0.051 | 0.159 | 4.33E−04 |

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, if needed, for safety factors. In this example the (a) Applied Safety Factor: Delivery System was “1”; the (b) Applied Safety Factor: Ingredients Modulating Skin Barrier to Enhance Bio-delivery was “5”; and the (c) Applied Safety Factor: Primed Immune Skin Condition Intended Use was “1”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category is not applicable. The safety adjusted sensitization values are shown below.

TABLE 18

| | | (b) Applied Safety | |
|---|---|---|---|
| | | Factor (“5”): | (c) Applied Safety |
| | | Ingredients | Factor (“1”): |
| Ingre- | (a) Applied Safety | Modulating Skin | Primed Immune |
| dient | Factor (“1”): | Barrier to Enhance | Skin Condition |
| No. | Delivery System | Bio-delivery | Intended Use |
| D1 | 3.30E−04 | 1.65E−03 | 1.65E−03 |
| D2 | 1.09E−04 | 5.44E−04 | 5.44E−04 |
| D3 | 4.26E−04 | 2.13E−03 | 2.13E−03 |
| D4 | 5.28E−05 | 2.64E−04 | 2.64E−04 |
| D5 | 1.30E−04 | 6.48E−04 | 6.48E−04 |
| D6 | 1.31E−04 | 6.54E−04 | 6.54E−04 |
| D7 | 6.26E−05 | 3.13E−04 | 3.13E−04 |
| D8 | 1.58E−04 | 7.89E−04 | 7.89E−04 |
| D9 | 5.43E−05 | 2.71E−04 | 2.71E−04 |
| D10 | 7.73E−05 | 3.87E−04 | 3.87E−04 |
| D11 | 9.55E−05 | 4.78E−04 | 4.78E−04 |
| D12 | 6.47E−05 | 3.23E−04 | 3.23E−04 |
| D13 | 2.22E−04 | 1.11E−03 | 1.11E−03 |
| D14 | 6.81E−05 | 3.40E−04 | 3.40E−04 |
| D15 | 7.39E−05 | 3.70E−04 | 3.70E−04 |
| D16 | 6.75E−05 | 3.37E−04 | 3.37E−04 |
| D17 | 1.70E−04 | 8.52E−04 | 8.52E−04 |
| D18 | 1.10E−04 | 5.50E−04 | 5.50E−04 |
| D19 | 9.15E−05 | 4.57E−04 | 4.57E−04 |
| D20 | 9.74E−05 | 4.87E−04 | 4.87E−04 |
| D21 | 1.73E−04 | 8.66E−04 | 8.66E−04 |
| D22 | 1.73E−04 | 8.66E−04 | 8.66E−04 |
| D23 | 1.41E−04 | 7.07E−04 | 7.07E−04 |
| D24 | 1.45E−04 | 7.26E−04 | 7.26E−04 |
| D25 | 1.63E−04 | 8.17E−04 | 8.17E−04 |
| D26 | 2.70E−04 | 1.35E−03 | 1.35E−03 |
| D27 | 6.06E−04 | 3.03E−03 | 3.03E−03 |
| D28 | 6.09E−04 | 3.05E−03 | 3.05E−03 |
| D29 | 4.33E−04 | 2.16E−03 | 2.16E−03 |

The order of the category of the applied safety factors was not critical. Indeed, the category of the applied safety factors may be provided in any desirable order.

The safety factor adjusted sensitization values were then summed, as shown in Table 19 below.

TABLE 19

| | Ingredient Real-World Application |
|---|---|
| Ingredient | Sensitization Values Adjusted for |
| No. | Intended Application And Safety Factors |
| D1 | 1.65E−03 |
| D2 | 5.44E−04 |
| D3 | 2.13E−03 |
| D4 | 2.64E−04 |
| D5 | 6.48E−04 |
| D6 | 6.54E−04 |
| D7 | 3.13E−04 |
| D8 | 7.89E−04 |
| D9 | 2.71E−04 |
| D10 | 3.87E−04 |
| D11 | 4.78E−04 |
| D12 | 3.23E−04 |
| D13 | 1.11E−03 |
| D14 | 3.40E−04 |
| D15 | 3.70E−04 |
| D16 | 3.37E−04 |
| D17 | 8.52E−04 |
| D18 | 5.50E−04 |
| D19 | 4.57E−04 |
| D20 | 4.87E−04 |
| D21 | 8.66E−04 |
| D22 | 8.66E−04 |
| D23 | 7.07E−04 |
| D24 | 7.26E−04 |
| D25 | 8.17E−04 |
| D26 | 1.35E−03 |
| D27 | 3.03E−03 |
| D28 | 3.05E−03 |
| D29 | 2.16E−03 |
| Total Sum: | 0.0265 |

As the calculated sensitization value of 0.0265 for the proposed formulation was less than a typical product sensitization value or threshold of 0.1, the proposed formulation is capable of application to the skin of users without or with just minimal testing as it is correlated with real-world data.

### EXAMPLE 5

A Leave-On Anti-Acne was formulated with the following ingredients as shown in Table 20 below:

TABLE 20

| Proposed Leave-On Anti-Acne | Ingredient |
|---|---|
| Formula Ingredient Composition | No. |
| Vehicle | E1 |
| Binder | E2 |
| Film Former | E3 |
| Skin conditioner | E4 |
| Skin conditioner | E5 |
| Astringent | E6 |
| Skin Conditioner | E7 |
| Solubilizer | E8 |
| Humectant | E9 |
| Vehicle | E10 |
| Fragrance | E11 |
| Skin conditioner | E12 |
| Humectant | E13 |
| Preservative | E14 |
| Vehicle | E15 |
| Skin conditioner | E16 |
| Buffering agent | E17 |
| Chelating agent | E18 |
| Skin conditioner | E19 |
| Solubilizer | E20 |
| Anti-acne agent | E21 |
| Film Former | E22 |
| Skin conditioner | E23 |
| Surfactant | E24 |
| Antimicrobial agent | E25 |
| Skin conditioner | E26 |
| Skin conditioner | E27 |
| Viscosity controlling agent | E28 |
| Skin conditioner | E29 |

All of the ingredients were used in ten or more personal care products. The number of tested formulas containing the above ingredients varied from 16 to 1,176. All of the ingredients were tested on 2,000 or more subjects. The total number of subjects tested in formulas containing the above ingredient varied from 2,519 to 187,429.

The ingredient sensitization for each of the ingredients is shown in Table 21 below. The upper confidence limit (UCL(p)) at 95% confidence level was calculated for each ingredient. A dosage density factor 1.54/565 (representing 1.54 g of daily dose over 565 cm² usage surface area from Table 1 above) was applied to the upper confidence limit for each ingredient. These values are shown below in Table 21 below.

TABLE 21

| Ingredient | Ingredient | Ingredient | Dosage Density Adjusted |
|---|---|---|---|
| No. | Sensitization (%) | UCL(p) | Ingredient Sensitization |
| E1 | 0.000 | 0.049 | 1.34E−04 |
| E2 | 0.000 | 0.055 | 1.50E−04 |
| E3 | 0.000 | 0.077 | 2.11E−04 |
| E4 | 0.039 | 0.185 | 5.03E−04 |
| E5 | 0.000 | 0.079 | 2.15E−04 |
| E6 | 0.040 | 0.185 | 5.03E−04 |
| E7 | 0.006 | 0.019 | 5.28E−05 |
| E8 | 0.011 | 0.026 | 7.02E−05 |
| E9 | 0.012 | 0.023 | 6.26E−05 |
| E10 | 0.014 | 0.065 | 1.76E−04 |
| E11 | 0.014 | 0.020 | 5.43E−05 |
| E12 | 0.017 | 0.080 | 2.19E−04 |
| E13 | 0.017 | 0.024 | 6.47E−05 |
| E14 | 0.017 | 0.025 | 6.81E−05 |
| E15 | 0.019 | 0.025 | 6.75E−05 |
| E16 | 0.021 | 0.099 | 2.71E−04 |
| E17 | 0.024 | 0.034 | 9.15E−05 |
| E18 | 0.024 | 0.036 | 9.74E−05 |
| E19 | 0.027 | 0.086 | 2.34E−04 |
| E20 | 0.028 | 0.052 | 1.41E−04 |
| E21 | 0.031 | 0.071 | 1.94E−04 |
| E22 | 0.037 | 0.116 | 3.16E−04 |
| E23 | 0.039 | 0.185 | 5.03E−04 |
| E24 | 0.040 | 0.188 | 5.12E−04 |
| E25 | 0.041 | 0.105 | 2.86E−04 |
| E26 | 0.050 | 0.130 | 3.54E−04 |
| E27 | 0.059 | 0.152 | 4.13E−04 |
| E28 | 0.026 | 0.043 | 1.18E−04 |
| E29 | 0.017 | 0.027 | 7.39E−05 |

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, if needed, for safety factors. In this example the (a) Applied Safety Factor: Delivery System was “1”; the (b) Applied Safety Factor: Ingredients Modulating Skin Barrier to Enhance Bio-delivery was “5”; and the (c) Applied Safety Factor: Primed Immune Skin Condition Intended Use was “2”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category is not applicable. The safety adjusted sensitization values are shown below.

TABLE 22

| | | (b) Applied Safety | |
|---|---|---|---|
| | | Factor (“5”): | (c) Applied Safety |
| | | Ingredients | Factor (“2”): |
| Ingre- | (a) Applied Safety | Modulating Skin | Primed Immune |
| dient | Factor (“1”): | Barrier to Enhance | Skin Condition |
| No. | Delivery System | Bio-delivery | Intended Use |
| E1 | 1.34E−04 | 6.71E−04 | 1.34E−03 |
| E2 | 1.50E−04 | 7.49E−04 | 1.50E−03 |
| E3 | 2.11E−04 | 1.05E−03 | 2.11E−03 |
| E4 | 5.03E−04 | 2.52E−03 | 5.03E−03 |
| E5 | 2.15E−04 | 1.08E−03 | 2.15E−03 |
| E6 | 5.03E−04 | 2.52E−03 | 5.03E−03 |
| E7 | 5.28E−05 | 2.64E−04 | 5.28E−04 |
| E8 | 7.02E−05 | 3.51E−04 | 7.02E−04 |
| E9 | 6.26E−05 | 3.13E−04 | 6.26E−04 |
| E10 | 1.76E−04 | 8.82E−04 | 1.76E−03 |
| E11 | 5.43E−05 | 2.71E−04 | 5.43E−04 |
| E12 | 2.19E−04 | 1.09E−03 | 2.19E−03 |
| E13 | 6.47E−05 | 3.23E−04 | 6.47E−04 |
| E14 | 6.81E−05 | 3.40E−04 | 6.81E−04 |
| E15 | 6.75E−05 | 3.37E−04 | 6.75E−04 |
| E16 | 2.71E−04 | 1.36E−03 | 2.71E−03 |
| E17 | 9.15E−05 | 4.57E−04 | 9.15E−04 |
| E18 | 9.74E−05 | 4.87E−04 | 9.74E−04 |
| E19 | 2.34E−04 | 1.17E−03 | 2.34E−03 |
| E20 | 1.41E−04 | 7.07E−04 | 1.41E−03 |
| E21 | 1.94E−04 | 9.68E−04 | 1.94E−03 |
| E22 | 3.16E−04 | 1.58E−03 | 3.16E−03 |
| E23 | 5.03E−04 | 2.52E−03 | 5.03E−03 |
| E24 | 5.12E−04 | 2.56E−03 | 5.12E−03 |
| E25 | 2.86E−04 | 1.43E−03 | 2.86E−03 |
| E26 | 3.54E−04 | 1.77E−03 | 3.54E−03 |
| E27 | 4.13E−04 | 2.07E−03 | 4.13E−03 |
| E28 | 1.18E−04 | 5.92E−04 | 1.18E−03 |
| E29 | 7.39E−05 | 3.7E−04 | 7.39E−04 |

The order of the category of the applied safety factors was not critical. Indeed, the category of the applied safety factors may be provided in any desirable order.

The safety factor adjusted sensitization values were then summed, as shown in Table 23 below.

TABLE 23

| | Ingredient Real-World Application |
|---|---|
| Ingredient | Sensitization Values Adjusted for |
| No. | Intended Application And Safety Factors |
| E1 | 1.34E−03 |
| E2 | 1.50E−03 |
| E3 | 2.11E−03 |
| E4 | 5.03E−03 |
| E5 | 2.15E−03 |
| E6 | 5.03E−03 |
| E7 | 5.28E−04 |
| E8 | 7.02E−04 |
| E9 | 6.26E−04 |
| E10 | 1.76E−03 |
| E11 | 5.43E−04 |
| E12 | 2.19E−03 |
| E13 | 6.47E−04 |
| E14 | 6.81E−04 |
| E15 | 6.75E−04 |
| E16 | 2.71E−03 |
| E17 | 9.15E−04 |
| E18 | 9.74E−04 |
| E19 | 2.34E−03 |
| E20 | 1.41E−03 |
| E21 | 1.94E−03 |
| E22 | 3.16E−03 |
| E23 | 5.03E−03 |
| E24 | 5.12E−03 |
| E25 | 2.86E−03 |
| E26 | 3.54E−03 |
| E27 | 4.13E−03 |
| E28 | 1.18E−03 |
| E29 | 7.39E−04 |
| Total Sum: | 0.0616 |

As the calculated sensitization value of 0.0616 for the proposed formulation was less than a typical product sensitization value or threshold of 0.1, the proposed formulation is capable of application to the skin of users without or with just minimal testing as it is correlated with real-world data.

### EXAMPLE 6

A Baby Wash (6 month) (Rinse Off) was formulated with the following ingredients as shown in Table 24 below:

TABLE 24

| Proposed Baby Wash (6 month) | |
|---|---|
| (Rinse Off) Formula | Ingredient |
| Ingredient Composition | No. |
| Viscosity Increasing agent | F1 |
| Surfactant | F2 |
| Skin conditioner | F3 |
| Surfactant | F4 |
| Preservative | F5 |
| Fragrance | F6 |
| Skin conditioner | F7 |
| Surfactant | F8 |
| Preservative | F9 |
| Preservative | F10 |
| pH Adjuster | F11 |
| Chelating Agent | F12 |
| Vehicle | F13 |

All of the ingredients were used in ten or more personal care products. The number of tested formulas containing the above ingredients varied from 16 to 1,176. All of the ingredients were tested on 2,000 or more subjects. The total number of subjects tested in formulas containing the above ingredient varied from 2,160 to 187,429.

The ingredient sensitization for each of the ingredients is shown in Table 25 below. The upper confidence limit (UCL(p)) at 95% confidence level was calculated for each ingredient. A dosage density factor 5.98/3500 (representing 5.98 g of daily dose over 3,500 cm² usage surface area from Table 1 above) was applied to the upper confidence limit for each ingredient. These values are shown below in Table 25 below.

TABLE 25

| Ingredient | Ingredient | Ingredient | Dosage Density Adjusted |
|---|---|---|---|
| No. | Sensitization (%) | UCL(p) | Ingredient Sensitization |
| F1 | 0.011 | 0.025 | 4.26E−05 |
| F2 | 0.015 | 0.028 | 4.84E−05 |
| F3 | 0.006 | 0.029 | 4.97E−05 |
| F4 | 0.015 | 0.047 | 8.00E−05 |
| F5 | 0.025 | 0.041 | 6.94E−05 |
| F6 | 0.014 | 0.020 | 3.40E−05 |
| F7 | 0.000 | 0.021 | 3.63E−05 |
| F8 | 0.016 | 0.042 | 7.14E−05 |
| F9 | 0.017 | 0.025 | 4.27E−05 |
| F10 | 0.010 | 0.021 | 3.53E−05 |
| F11 | 0.024 | 0.034 | 5.73E−05 |
| F12 | 0.000 | 0.139 | 2.37E−04 |
| F13 | 0.019 | 0.025 | 4.23E−05 |

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, if needed, for safety factors. In this example the (a) Applied Safety Factor: Delivery System was “1”; the (b) Applied Safety Factor: Ingredients Modulating Skin Barrier to Enhance Bio-delivery was “1”; and the (c) Applied Safety Factor: Primed Immune Skin Condition Intended Use was “1”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category is not applicable. The safety adjusted sensitization values are shown below.

TABLE 26

| | | (b) Applied Safety | |
|---|---|---|---|
| | | Factor (“1”): | (c) Applied Safety |
| | | Ingredients | Factor (“1”): |
| Ingre- | (a) Applied Safety | Modulating Skin | Primed Immune |
| dient | Factor (“1”): | Barrier to Enhance | Skin Condition |
| No. | Delivery System | Bio-delivery | Intended Use |
| F1 | 4.26E−05 | 4.26E−05 | 4.26E−05 |
| F2 | 4.84E−05 | 4.84E−05 | 4.84E−05 |
| F3 | 4.97E−05 | 4.97E−05 | 4.97E−05 |
| F4 | 8.00E−05 | 8.00E−05 | 8.00E−05 |
| F5 | 6.94E−05 | 6.94E−05 | 6.94E−05 |
| F6 | 3.40E−05 | 3.40E−05 | 3.40E−05 |
| F7 | 3.63E−05 | 3.63E−05 | 3.63E−05 |
| F8 | 7.14E−05 | 7.14E−05 | 7.14E−05 |
| F9 | 4.27E−05 | 4.27E−05 | 4.27E−05 |
| F10 | 3.53E−05 | 3.53E−05 | 3.53E−05 |
| F11 | 5.73E−05 | 5.73E−05 | 5.73E−05 |
| F12 | 2.37E−04 | 2.37E−04 | 2.37E−04 |
| F13 | 4.23E−05 | 4.23E−05 | 4.23E−05 |

The order of the category of the applied safety factors was not critical. Indeed, the category of the applied safety factors may be provided in any desirable order.

The safety factor adjusted sensitization values were then summed, as shown in Table 27 below.

TABLE 27

| | Ingredient Real-World Application |
|---|---|
| Ingredient | Sensitization Values Adjusted for |
| No. | Intended Application And Safety Factors |
| F1 | 4.26E−05 |
| F2 | 4.84E−05 |
| F3 | 4.97E−05 |
| F4 | 8.00E−05 |
| F5 | 6.94E−05 |
| F6 | 3.40E−05 |
| F7 | 3.63E−05 |
| F8 | 7.14E−05 |
| F9 | 4.27E−05 |
| F10 | 3.53E−05 |
| F11 | 5.73E−05 |
| F12 | 2.37E−04 |
| F13 | 4.23E−05 |
| Total Sum: | 0.000804 |

As the calculated sensitization value of 0.000804 for the proposed formulation was less than a typical product sensitization value or threshold of 0.1, the proposed formulation is capable of application to the skin of users without or with just minimal testing as it is correlated with real-world data.

### Comparative Example 7

A Face Mask having anti-acne agent was formulated with the following ingredients as shown in Table 28 below:

TABLE 28

| Proposed Face Mask anti-acne | Ingredient |
|---|---|
| Formula Ingredient Composition | No. |
| Occlusive Absorbent | G1 |
| Occlusive Absorbent | G2 |
| Preservative | G3 |
| Opacifying agent | G4 |
| Fragrance | G5 |
| Cooling agent | G6 |
| Humectant | G7 |
| Vehicle | G8 |
| Buffering agent | G9 |
| pH adjuster | G10 |
| pH adjuster | G11 |
| Chelating agent | G12 |
| Viscosity increasing agent | G13 |
| surfactant | G14 |
| Anti-acne agent | G15 |

The number of tested formulas containing the above ingredients varied from 12 to 1,176. The total number of subjects tested in formulas containing the above ingredient varied from 2,006 to 187,429.

The ingredient sensitization for each of the ingredients is shown in Table 29 below. The upper confidence limit (UCL(p)) at 95% confidence level was calculated for each ingredient. A dosage density factor 1.54/565 (representing 1.54 g of daily dose over 565 cm² usage surface area from Table 1 above) was applied to the upper confidence limit for each ingredient. These values are shown below in Table 29 below.

TABLE 29

| Ingredient | Ingredient | Ingredient | Dosage Density Adjusted |
|---|---|---|---|
| No. | Sensitization (%) | UCL(p) | Ingredient Sensitization |
| G1 | 0.000 | 0.148 | 4.00E−04 |
| G2 | 0.000 | 0.148 | 4.00E−04 |
| G3 | 0.008 | 0.019 | 5.13E−05 |
| G4 | 0.009 | 0.020 | 5.46E−05 |
| G5 | 0.014 | 0.020 | 5.43E−05 |
| G6 | 0.014 | 0.067 | 1.84E−04 |
| G7 | 0.017 | 0.024 | 6.47E−05 |
| G8 | 0.019 | 0.025 | 6.75E−05 |
| G9 | 0.019 | 0.038 | 1.04E−04 |
| G10 | 0.021 | 0.032 | 8.65E−05 |
| G11 | 0.024 | 0.034 | 9.15E−05 |
| G12 | 0.024 | 0.036 | 9.74E−05 |
| G13 | 0.026 | 0.043 | 1.18E−04 |
| G14 | 0.052 | 0.247 | 6.72E−04 |
| G15 | 0.55 | 2.2 | 6.00E−03 |

Moreover, in this example the ingredient sensitization values and the upper confidence limits for each ingredient were not adjusted for the weight composition of the ingredients in the proposed formulation. Nevertheless, if desired, these values and limits may be adjusted for the weight composition of the ingredients in the proposed formulation.

The dosage density adjusted ingredient sensitization values for each ingredient were adjusted, if needed, for safety factors. In this example the (a) Applied Safety Factor: Delivery System was “5”; the (b) Applied Safety Factor: Ingredients Modulating Skin Barrier to Enhance Bio-delivery was “5”; and the (c) Applied Safety Factor: Primed Immune Skin Condition Intended Use was “2”. The first applied safety factor was multiplied across the Dosage Density Adjusted Ingredient Sensitization values, then the second applied safety factor was multiplied across the sensitization values adjusted by the first applied safety factor; and then the third applied safety factor was multiplied across the sensitization values adjusted by the second safety factor. A safety factor of “1” means that no adjustment was being made to the sensitization values, i.e., the particular safety category is not applicable. The safety adjusted sensitization values are shown below.

TABLE 30

| | | (b) Applied Safety | |
|---|---|---|---|
| | | Factor (“5”): | (c) Applied Safety |
| | | Ingredients | Factor (“2”): |
| Ingre- | (a) Applied Safety | Modulating Skin | Primed Immune |
| dient | Factor (“5”): | Barrier to Enhance | Skin Condition |
| No. | Delivery System | Bio-delivery | Intended Use |
| G1 | 2.02E−03 | 1.01E−02 | 2.02E−02 |
| G2 | 2.02E−03 | 1.01E−02 | 2.02E−02 |
| G3 | 2.56E−04 | 1.28E−03 | 2.56E−03 |
| G4 | 2.73E−04 | 1.36E−03 | 2.73E−03 |
| G5 | 2.71E−04 | 1.36E−03 | 2.71E−03 |
| G6 | 9.19E−04 | 4.59E−03 | 9.19E−03 |
| G7 | 3.23E−04 | 1.62E−03 | 3.23E−03 |
| G8 | 3.37E−04 | 1.69E−03 | 3.37E−03 |
| G9 | 5.18E−04 | 2.59E−03 | 5.18E−03 |
| G10 | 4.32E−04 | 2.16E−03 | 4.32E−03 |
| G11 | 4.57E−04 | 2.29E−03 | 4.57E−03 |
| G12 | 4.87E−04 | 2.44E−03 | 4.87E−03 |
| G13 | 5.92E−04 | 2.96E−03 | 5.92E−03 |
| G14 | 3.36E−03 | 1.68E−02 | 3.36E−02 |
| G15 | 3.00E−02 | 1.50E−01 | 3.0E−01 |

The order of the category of the applied safety factors was not critical. Indeed, the category of the applied safety factors may be provided in any desirable order.

The safety factor adjusted sensitization values were then summed, as shown in Table 31 below.

TABLE 31

| | Ingredient Real-World Application |
|---|---|
| Ingredient | Sensitization Values Adjusted for |
| No. | Intended Application And Safety Factors |
| G1 | 2.02E−02 |
| G2 | 2.02E−02 |
| G3 | 2.56E−03 |
| G4 | 2.73E−03 |
| G5 | 2.71E−03 |
| G6 | 9.19E−03 |
| G7 | 3.23E−03 |
| G8 | 3.37E−03 |
| G9 | 5.18E−03 |
| G10 | 4.32E−03 |
| G11 | 4.57E−03 |
| G12 | 4.87E−03 |
| G13 | 5.92E−03 |
| G14 | 3.36E−02 |
| G15 | 3.00E−01 |
| Total Sum: | 0.422 |

The calculated sensitization value of 0.422 for the proposed formulation was greater than a typical product sensitization value or threshold of 0.1. The proposed formulation would likely require product sensitization testing. Alternatively, reformulation may also be considered.

While various embodiments and aspects of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

1. A method of predictively assessing skin sensitization of a personal care product formulation including a plurality of ingredients, the method comprising:
calculating an upper confidence limit at a desired confidence level from multiple physical tests for skin sensitization of each of the ingredients in the personal care product formulation in order to define calculated skin sensitization values for the respective ingredients;
applying at least one safety factor to at least one of the calculated skin sensitization values for the ingredients to define adjusted skin sensitization values for the respective ingredients;
summing the adjusted skin sensitization values of the ingredients to define a total sensitization of the personal care product formulation; and
comparing the total sensitization to a target product sensitization for the personal care product formulation, wherein the product formulation for the personal care product formulation does not require further testing and is capable of application to skin by users when the total sensitization is less than the target product sensitization.

2. The method of claim 1, wherein the skin sensitization of each of the ingredients is defined as the percentage of subjects having an allergic response to physical allergic tests.

3. The method of claim 2, wherein the physical allergic tests are human repeat insult tests.

4. The method of claim 1, further comprising:
setting an initial sensitization target for the personal care product formulation; and
obtaining the skin sensitization of each of the ingredients from a computer database having multiple test results for each ingredient.

5. The method of claim 4, wherein the initial sensitization target is about 0.2 percent or less than 0.2 percent.

6. The method of claim 1, wherein the target product sensitization is about 0.1 percent or less than 0.1 percent.

7. The method of claim 1, further comprising selecting the ingredients that have used in multiple personal care products.

8. The method of claim 1, wherein the step of selecting ingredients further comprises selecting ingredients that have used in about ten or more than ten different personal care products.

9. The method of claim 1, further comprising selecting the ingredients that have been tested on a plurality of subjects.

10. The method of claim 9, wherein the plurality of subjects is about 2000 subjects or greater than 2,000.

11. The method of claim 1, wherein the desired confidence level is at least 95%.

12. The method of claim 1, wherein the multiple physical tests comprise tests on about ten or more than ten different personal care products, and tests on about 2000 subjects or greater than 2000 subjects.

13. The method of claim 1, further comprising applying a product usage factor to the calculated skin sensitization values for the ingredients; wherein the product usage factor is based on an estimated amount of product to be used and an estimated area of product use.

14. The method of claim 13, wherein the product usage factor is a ratio of an estimated daily use in grams over an estimated application surface area in square-centimeters.

15. The method of claim 1, wherein the step of applying at least one safety factor comprises multiplying the at least one calculated skin sensitization value for at least one of the ingredients by the at least one safety factor, or the step of applying the at least one safety factor comprises multiplying the adjusted skin sensitization values for each of the ingredients by the at least safety factor.

16. (canceled).

17. The method of claim 15, wherein the at least one safety factor has a value of greater than one.

18. The method of claim 1, wherein the at least one safety factor is selected from safety factors selected from the group consisting of factors based on (a) impairment to skin barrier function, (b) based on product delivery effecting skin occlusion and/or based on occlusive nature application site, (c) based potential for pre-conditioned primed immune skin having potential for activated inflammatory response, and combinations thereof.

19. The method of claim 18, wherein, if applied, the safety factor based on impairment to skin barrier function is about 5 or greater than 5, the safety factor based on product delivery effecting skin occlusion and/or based on occlusive nature application site is about 5 or greater than 5, and the safety factor based potential for pre-conditioned primed immune skin having potential for activated inflammatory response is about 2 or greater than 2.

20. A system to predictively assess skin sensitization of a personal care product formulation including a plurality of ingredients, the system comprising:
a computing device;
a memory storing instructions that, when executed by the computing device, cause the computing device to perform operations comprising:
calculating an upper confidence limit at a desired confidence level from multiple tests for skin sensitization of each of the ingredients in the personal care product formulation in order to define calculated skin sensitization values for respective ingredients;
applying at least one safety factor to the calculated skin sensitization values for the ingredients to define adjusted skin sensitization values for the respective ingredients;
summing the adjusted skin sensitization values of the ingredients to define a total sensitization of the personal care product formulation; and
comparing the total sensitization to a target product sensitization for the personal care product formulation, wherein the product formulation does not require further testing and is capable of application to skin by users when the total sensitization is less than the target product sensitization.

21. A method of preparing a personal care product comprising:
selecting a plurality ingredients for use in a personal care product formulation;
calculating an upper confidence limit at a desired confidence level from multiple physical tests for skin sensitization of each of the ingredients in the personal care product formulation in order to define calculated skin sensitization values for the respective ingredients;
applying at least one safety factor to at least one of the calculated skin sensitization values for the ingredients to define adjusted skin sensitization values for the respective ingredients;
summing the adjusted skin sensitization values of the ingredients to define a total sensitization of the personal care product formulation;
comparing the total sensitization to a target product sensitization for the personal care product formulation; and
if the total sensitization is less that the target product sensitization, combining the ingredients to form the personal care product, wherein the product formulation for the personal care product formulation does not require further testing and is capable of application to skin by users.
